(19) United States
(12) Patent Application Publication
(10) Pub. No.: US 2024/0273724 A1
(43) Pub. Date: Aug. 15, 2024

(54) CONTACTLESS INTOXICATION DETECTION AND METHODS AND SYSTEMS THEREOF

(71) Applicant: DISPENSION INDUSTRIES INCORPORATED, Dartmouth (CA)

(72) Inventors: Georgia KOUKIOU, Patras (GR); Vassilis ANASTASSOPOULOS, Patras (GR); Carl GOODWIN, Truro (CA); Matthew MICHAELIS, Dartmouth (CA); Corey YANTHA, Dartmouth (CA); Rob MCINTYRE, Vancouver (CA)

(21) Appl. No.: 18/568,742

(22) PCT Filed: Jun. 10, 2022
(86) PCT No.: PCT/CA2022/050936

§ 371 Date: Dec. 8, 2023

Related U.S. Application Data
(60) Provisional application No. 63/216,916, filed on Jun. 30, 2021.

(30) Foreign Application Priority Data
Jun. 11, 2021 (EP) 21386034.9

(51) Int. Cl. G06T 7/00; A61B 5/01; A61B 5/18; G06T 3/40; G06V 10/80; G06V 10/82; G06V 40/16; G06V 40/18

(52) U.S. Cl. CPC G06T 7/0014; A61B 5/015; A61B 5/18; G06T 3/40; G06V 10/806; G06V 10/82; G06V 40/168; G06V 40/18; G06T 2207/10048; G06T 2207/20084; G06T 2207/30041; G06T 2207/30101; G06T 2207/30201

(57) ABSTRACT

The present disclosure provide non-invasive and contactless intoxication detection methods and systems. For example, there is provided a non-invasive method for assessing the intoxication status or level of an individual, the method comprising: receiving a thermographic image comprising a face or facial features of the individual, performing pre-processing of the thermographic image to provide a pre-processed image, identifying a face portion comprising the face or facial features in the pre-processed image, and analyzing the face portion using an intoxication assessment method to assess the intoxication status. Systems for performing the disclosed methods are also provided.

### CROSS-REFERENCE TO RELATED APPLICATIONS

This application is a U.S. national phase of PCT International Patent Application No. PCT/CA2022/050936, filed Jun. 10, 2022, which claims priority to and benefit of European Patent Application No. 21386034.9 filed on Jun. 11, 2021 and U.S. Patent Application No. 63/216,916 filed on Jun. 30, 2021, each of which are hereby incorporated by reference in their entireties.

### TECHNICAL FIELD

The present disclosure generally relates to a non-invasive and contactless intoxication detection methods and systems. In particular, the present disclosure provides methods and systems for assessing the intoxication status or level of an individual.

### BACKGROUND

The subject of persons performing complex tasks while intoxicated, such as driving cars, operating machinery or piloting passenger conveyances, are well documented; as are the potential for social problems such as at alcoholic sales venues.

At times it is easy to detect intoxicated individuals and to take steps to mitigate any associated risks, but at other times it is not. Some individuals do not appear intoxicated, when in fact they are over a legal limit or otherwise exceed some acceptable threshold of intoxication for the task or service they are providing, or the venue they are attending.

Current methods for detecting such persons are quite invasive, requiring the collection of breath, urine or blood. Aside from being distressing to the subject, these methods are subject to legal, ethical and practical difficulties in the field—for example, during a traffic stop.

It is therefore of interest to various enforcement groups to be able to identify intoxicated individuals from a distance and by a non-invasive means. If this can be achieved then a new system for preventing harm due to intoxicated individuals can be operationalised, saving society the costs associated with such persons when their indiscretions result in actual harm to property and others.

Therefore, a need exists for improved and non-invasive methods and systems for assessing the intoxication status of an individual.

### SUMMARY

The present disclosure relates to a non-invasive, contactless method for assessing the intoxication status or level of an individual. In select embodiments, various advantages may be provided over prior technologies, including for example non-invasiveness, rejection of false positives, pre-processing of thermographic images for increased accuracy and computational efficiency, assessment that is independent of a priori data on a given individual, signalling means for use by other systems; a system of logging that is capable of standing up to legal challenge, and a reliable scoring method. Taken together, the present disclosure provides methods and systems that can not only detect intoxicated individuals accurately and reliably, but is also capable of providing or timely signalling of such as well as an encapsulating record of facts that can be relied on in case management.

In a broad aspect, a method for assessing an intoxication status of an individual includes the steps of receiving a thermographic image including a face or facial features of the individual, performing pre-processing of the thermographic image to provide a pre-processed image, identifying a face portion including the face or facial features in the pre-processed image, and analyzing the face portion using an intoxication assessment method to assess the intoxication status.

In an embodiment disclosed herein, the thermographic image includes faces or facial features of other persons and the step of identifying the face portion includes isolating the face or facial features of the individual.

In an embodiment disclosed herein, the pre-processing includes reducing the thermographic image to a single channel. In an embodiment, the single channel is brightness. In other embodiments, the pre-processing may include reducing the thermographic image into more than one channel, such as for example and without limitation, individually into a red channel, green channel, blue channel, or an alpha channel, or any combination thereof, including all of these channels (i.e. RGB+A).

In an embodiment disclosed herein, the pre-processing includes removing data from the thermographic image for temperatures outside of a temperature range.

In an embodiment disclosed herein, the temperature range is between 17 degrees Celsius and 47 degrees Celsius.

In an embodiment disclosed herein, the temperature range is between 32 degrees Celsius and 42 degrees Celsius.

In an embodiment disclosed herein, the pre-processing includes reducing a resolution of the thermographic image.

In an embodiment disclosed herein, the step of identifying the face portion uses a convolutional neural network.

In an embodiment disclosed herein, the step of identifying the face portion uses a Haar Cascade.

In an embodiment disclosed herein, the method includes an additional step of detecting an obstruction to the face portion.

In an embodiment disclosed herein, the obstruction is one or more of a beard, a mask, a moustache, a hat, a pair of glasses, a pair of sunglasses, a neck brace, an eye patch, a medical dressing, a turtle neck shirt, a tattoo, a pair of headphones, and a pair of ear muffs.

In an embodiment disclosed herein, the intoxication status includes intoxicated and non-intoxicated.

In an embodiment disclosed herein, the intoxication assessment method includes identifying a plurality of points in the face portion to provide a facial feature vector, comparing the facial feature vector with other facial feature vectors for other thermographic images to identify differences therebetween, and assessing the intoxication status by analyzing the differences.

In an embodiment disclosed herein, the plurality of points is at least twenty.

In an embodiment disclosed herein, the intoxication assessment method includes identifying at least two regions in the face portion, and determining a face temperature difference between the two regions for assessing the intoxication status.

In an embodiment disclosed herein, the two regions include a nose area and a forehead area, the nose area including an image of a nose of the individual and the forehead area including an image of a forehead of the individual.

In an embodiment disclosed herein, the intoxication assessment method includes identifying an eye region in the face portion, the eye region including an image of eyes of the individual, identifying a sclera region and an iris region within the eye region, the sclera region including an image of a sclera of the individual and the iris region including an image of an iris of the individual, and determining an eye temperature difference between the sclera region and the iris region for assessing the intoxication status.

In an embodiment disclosed herein, the intoxication assessment method includes using a trained neural network to analyze the face portion to assess the intoxication status.

In an embodiment disclosed herein, the intoxication assessment method includes identifying a high correlation area in the face portion, and using a trained neural network to analyze the high correlation area to assess the intoxication status.

In an embodiment disclosed herein, the high correlation area is a nose area, a mouth area or a combination thereof, wherein the nose area includes an image of a nose of the individual and a mouth area includes an image of a mouth of the individual.

In an embodiment disclosed herein, the intoxication assessment method includes identifying blood vessel in the face portion, and analyzing the blood vessels to determine changes or differences in blood vessel activity to assess the intoxication status.

In an embodiment disclosed herein, the step of analyzing blood vessel locations includes applying a nonlinear anisotropic diffusion and a top-hat transformation of the face portion.

In an embodiment disclosed herein, the step of analyzing blood vessel locations includes using image processing to perform image transformations, convolutions, edge detections, or related means to determine changes or differences in blood vessel activity.

In an embodiment disclosed herein, the intoxication assessment method includes identifying and analyzing one or more isothermal regions in the face portion to assess the intoxication status.

In an embodiment disclosed herein, the step of identifying and analyzing one or more isothermal regions includes determining a shape and a size of the isothermal regions.

In an embodiment disclosed herein, at least of one of the isothermal regions is a forehead region of the face portion including an image of a forehead of the individual, and wherein when the forehead region is thermally isolated from a remainder of the face portion, the intoxication status is intoxicated.

In an embodiment disclosed herein, the intoxication assessment method includes using Markov chains or Bayesian networks for modeling statistical behaviour of pixels in a forehead region of the face portion, wherein the forehead region includes an image of a forehead of the individual.

In an embodiment disclosed herein, the intoxication assessment method includes identifying local difference patterns in the face portion to assess the intoxication status.

In an embodiment disclosed herein, the intoxication assessment method includes feature fusion analysis to fuse dissimilar features of the face portion using neural networks to assess the intoxication status.

In an embodiment disclosed herein, the method for assessing an intoxication status of an individual includes the step of analyzing the face portion using one or more additional intoxication assessment methods to confirm the intoxication status.

In an embodiment disclosed herein, the intoxication status relates to intoxication by alcohol.

In a broad aspect, a system for assessing an intoxication status of an individual includes a computer configured to perform pre-processing of one or more thermographic images including a face or facial features of the individual, identifying a face portion of the thermographic images including the face or facial features, and analyzing the face portion using at least one intoxication assessment method to assess the intoxication status.

In an embodiment disclosed herein, the system includes a device including an infrared camera to obtain the one or more thermographic images, an input interface for receiving instructions, and an output interface for displaying the intoxication status, wherein the device is connected to the computer for communication therebetween.

In an embodiment disclosed herein, the computer and the device include network communications systems for communicating instructions, the one or more thermographic images, and the intoxication status.

In an embodiment disclosed herein, the output interface includes a screen that graphically presents the one or more thermographic images, annotations to the one or more thermographic images, graphs, other suitable data, or any combination thereof to communicate the assessment of the intoxication status.

In an embodiment disclosed herein, the output interface includes a matrix display, LCD, LED, buzzer, speaker, light, numerical value, picture, image, other visual or audio reporting means, or any combination thereof to communicate the assessment of the intoxication status.

In an embodiment disclosed herein, the device includes one or more sensors or data inputs for recording date, time, position, orientation, temperature, humidity or other geo-temporal and physical conditions at the time of obtaining the one or more thermographic images.

In an embodiment disclosed herein, the device includes one or more accessory components to ascertain the identity of an operator and/or the individual by manual input, swipe card, barcode, biometric, RFID, NFC or other identifying means.

In an embodiment disclosed herein, the computer includes an external communication component that is capable of communicating with other systems for receiving information, storage, or further processing.

In an embodiment disclosed herein, the system is capable of a two-way communication with one or more remote storage systems, the two-way communication performing encrypted communications in a manner that guarantees the authenticity of those data stored on the computer, the device, the remote system(s), or any combination thereof.

In an embodiment disclosed herein, the device is portable.

In an embodiment disclosed herein, the device is handheld.

In an embodiment disclosed herein, the device is a kiosk.

In an embodiment disclosed herein, the kiosk is a self-service intoxicant dispensing kiosk.

In an embodiment disclosed herein, the device is a stand-alone device for use as a non-invasive screening tool for determining whether the individual is permitted to operate a vehicle or machine.

In an embodiment disclosed herein, the device is configured for integration into a vehicle or machine, and when integrated can prevent the individual from operating the vehicle or machine based on the assessment of the intoxication status.

In an embodiment disclosed herein, the intoxication status relates to intoxication by alcohol.

In a broad aspect, one or more non-transitory computer-readable storage devices including computer-executable instructions for providing an assessment of an intoxication status of an individual, wherein the instructions, when executed, cause a processing structure to perform actions including receiving a thermographic image including a face or facial features of the individual, performing pre-processing of the thermographic image to provide a pre-processed image, identifying a face portion including the face or facial features in the pre-processed image, and analyzing the face portion using an intoxication assessment method to assess the intoxication status.

In an embodiment disclosed herein, the one or more non-transitory computer-readable storage devices, wherein the instructions, when executed, cause the processing structure to perform further actions relating to performing pre-processing including identifying the face portion by isolating the face or facial features of the individual, wherein the thermographic further includes faces or facial features of other persons.

In an embodiment disclosed herein, the one or more non-transitory computer-readable storage devices, wherein the instructions, when executed, cause the processing structure to perform further actions relating to performing pre-processing including reducing the thermographic image to a single channel.

In an embodiment disclosed herein, the one or more non-transitory computer-readable storage devices, wherein the instructions, when executed, cause the processing structure to perform further actions relating to performing pre-processing including removing data from the thermographic image for temperatures outside of a temperature range.

In an embodiment disclosed herein, the one or more non-transitory computer-readable storage devices, wherein the instructions, when executed, cause the processing structure to perform further actions relating to performing pre-processing including reducing a resolution of the thermographic image.

In an embodiment disclosed herein, the one or more non-transitory computer-readable storage devices, wherein the instructions, when executed, cause the processing structure to perform further actions relating to identifying the face portion using a convolutional neural network.

In an embodiment disclosed herein, the one or more non-transitory computer-readable storage devices, wherein the instructions, when executed, cause the processing structure to perform further actions relating to identifying the face portion using a Haar Cascade.

In an embodiment disclosed herein, the one or more non-transitory computer-readable storage devices, wherein the instructions, when executed, cause the processing structure to perform further actions relating to performing pre-processing including detecting an obstruction to the face portion.

In an embodiment disclosed herein, the one or more non-transitory computer-readable storage devices, wherein the instructions, when executed, cause the processing structure to perform further actions relating to performing of intoxication assessment methods.

Other aspects and embodiments of the disclosure are evident in view of the detailed description provided herein.

### BRIEF DESCRIPTION OF THE DRAWINGS

Further advantages, permutations and combinations of the invention will now appear from the above and from the following detailed description of the various particular embodiments of the invention taken together with the accompanying drawings, each of which are intended to be non-limiting, in which:

FIG. 1 is an image depicting exemplary monitoring of temperature changes with the consumption of alcohol based on the selection of points on the face.

FIG. 2 is a graph depicting exemplary clusters (16) from 8 persons in the 2-D space and showing that the clusters move towards the same direction with intoxication. The two most important directions correspond to the first two largest eigenvalues. This can be referred to as the “drunk” space.

FIG. 3 is an image depicting a face partitioned into a matrix of 8×5 squared regions. The thermal difference between these regions is monitored during alcohol consumption.

FIG. 4 shows three different matrices: (A) for an exemplary non-intoxicated person, (B) for an exemplary intoxicated person, and (C) showing the difference of the matrices of panels A and B (values normalized to full grayscale). In the panel C matrix, white pixels indicate the coordinates of the squared regions which present large changes in their thermal difference.

FIG. 5 are images showing regions that present the largest change in thermal differences for two different persons. For person A (panel A), eight squared regions on the forehead present thermal differences with respect to three squared regions around mouth. For person B (panel B), six squared regions on the forehead present thermal differences with respect to five squared regions around mouth.

FIG. 6 shows exemplary thermal images of the eyes of a non-intoxicated person (panel A) and an intoxicated person (panel B).

FIG. 7 shows two images, whereby panel A is an image obtained after applying anisotropic diffusion on the image of the intoxicated person shown in FIG. 1 and panel B shows the corresponding vessels extracted using top-hat transformation.

FIG. 8 depicts binary images obtained using a threshold equal to 100. Panel A shows a non-intoxicated individual and panel B shows an intoxicated individual. Vessels on the intoxicated individual are more distinct compared to those on the non-intoxicated individual.

FIG. 9 depicts isothermal regions, where panel A shows eight equal in length-width segments of the histogram (0-255) and panel B is an arbitrary determination based on the minima of the histogram.

FIG. 10 shows images for a non-intoxicated person and an intoxicated person. Panel A: For the non-intoxicated person the forehead lies in the same isothermal region together with other locations of the face. Panel B: The forehead lies in a different isothermal region than the rest of the face for the intoxicated person.

FIG. 11 are matrices for two different individuals, showing that the employed neural networks converge at areas corresponding to the forehead, the nose, and the mouth, which were found desirable for intoxication discrimination.

FIG. 12 shows the results obtained when a neural structure was trained using data from a first person and tested using the data from a second person. In panel A, the face of the second person is depicted largely as a black matrix, indicating a non-intoxicated person. In panel B, the face of the second person is depicted more by a white matrix, indicating an intoxicated person.

FIG. 13 shows a region of the forehead of an individual where the Markov properties of the pixels are studied. Panel A shows the entire face. Panel B is an enlargement of the forehead.

FIG. 14 is a scatter plot showing clusters of three individuals (diamond, circle, square) showing non-intoxicated (hollow) and intoxicated (solid) in the feature space. Units on the axis represent the values of the corresponding features (eigenvalues).

FIG. 15 is a graph of correctly classified 32D-vectors for each person either non-intoxicated (solid line) or intoxicated (dotted line). A total of 50 vectors correspond to each participant (sober or drunk).

FIG. 16 is a flowchart of an embodiment of a method of assessing an intoxication status of an individual.

FIG. 17 is a schematic diagram of a computerized system for assessing intoxication status of an individual, according to some embodiments of the present disclosure.

FIG. 18 is a schematic diagram showing a simplified hardware structure of a computing device of the system for assessing intoxication status of an individual of FIG. 17.

FIG. 19 a schematic diagram showing a simplified software architecture of a computing device of the system for assessing intoxication status of an individual of FIG. 17.

### DETAILED DESCRIPTION

Unless otherwise defined, all technical and scientific terms used herein generally have the same meaning as commonly understood by one of ordinary skill in the art to which this disclosure pertains. Exemplary terms are defined below for ease in understanding the subject matter of the present disclosure.

### Definitions

The term “a” or “an” refers to one or more of that entity; for example, “a computing element” refers to one or more computing elements or at least one computing element. As such, the terms “a” (or “an”), “one or more” and “at least one” are used interchangeably herein. In addition, reference to an element or feature by the indefinite article “a” or “an” does not exclude the possibility that more than one of the elements or features are present, unless the context clearly requires that there is one and only one of the elements. Furthermore, reference to a feature in the plurality (e.g. computing elements), unless clearly intended, does not mean that the systems or methods disclosed herein must comprise a plurality.

“About”, when referring to a measurable value such as an angle, a dimension, and the like, is meant to encompass variations of ±10%, ±5%, ±1%, ±0.5% or ±0.1% of the specified amount. When the value is a whole number, the term about is meant to encompass decimal values, as well the degree of variation just described. It is to be understood that such a variation is always included in any given value provided herein, whether or not it is specifically referred to.

“And/or” refers to and encompasses any and all possible combinations of one or more of the associated listed items (e.g. one or the other, or both), as well as the lack of combinations when interrupted in the alternative (or).

“Comprise” as is used in this description and in the claims, and its conjugations, is used in its non-limiting sense to mean that items following the word are included, but items not specifically mentioned are not excluded.

### Non-Invasive Intoxication Detection

The present disclosure relates to non-invasive, contactless methods for assessing the intoxication status or level of an individual. In select embodiments, various advantages may be provided over prior technologies.

As one example, embodiments of the methods and systems disclosed herein are non-invasive and capable of being used in a contactless manner.

As another, embodiments of the methods and systems disclosed comprise suitable pre-processing, which allow the efficient and effective use of thermographic images obtained under real world conditions.

As another, embodiments of the methods and systems disclosed are capable of reducing the incidence of false positives regarding intoxication status.

As another, embodiments of the methods and systems disclosed herein are capable of providing assessment of intoxication status or level independent of a priori data on a given individual, meaning that data for the tested individual in a non-intoxicated state is not required. The methods and systems of the present disclosure are capable of detecting an intoxicated individual without a “before” and an “after” image.

As another, embodiments of the methods and systems disclosed herein provide a signalling means for use by other systems.

As another, embodiments of the methods and systems disclosed herein provide a system of logging that is capable of standing up to legal challenge. Thus, the validity and enforceability of the results obtained by the methods and systems herein may be quite desirable to law enforcement agencies.

As another, embodiments of the methods and systems disclosed herein provide a reliable scoring method, such as the methods and systems being reproducible in the results provided.

Taken together, the present disclosure provides methods and systems that can not only detect intoxicated individuals accurately and reliably, but is also capable of providing or timely signalling of such as well as an encapsulating record of facts that can be relied on in case management.

The methods and systems of the present disclosure use an infrared (IR) or thermal imaging camera to photograph, and a system to then process and analyze the face of a potentially intoxicated person. The thermal signature is used to detect intoxication. The contactless intoxication detection of the present disclosure is based on using thermal cameras to map the thermal distribution on the face of individuals, and processing these images to make an intoxication assessment. The assessment may be done independent of any a priori data regarding the tested individual.

It is axiomatic that intoxicated individuals can appear “flushed” and this effect can lend itself well to detection by IR cameras. However, the same can be said for individuals with a fever. Accordingly, a key problem is in discriminating between those individuals who are actually intoxicated, as opposed to those individuals suffering from some other physiological condition. The present disclosure is advantageous in this respect.

A problem that has been overcome in the methods and systems disclosed herein was developing each aspect independently (e.g. aspects (i)-(ix) herein) and fusing the various aspects together with a weighting assigned to each based on the information available in the thermal image. For example, if the person being assessed is wearing glasses, the feature that assesses the thermal patterns in the eyes may be less heavily weighted than the other features. Advantageously, by the methods herein, this assessment can be performed with no a priori knowledge of the individual being tested.

### Pre-Processing of Thermographic Images

Embodiments of non-invasive methods disclosed herein for assessing the intoxication status or level of an individual are generally designed and configured to analyze thermographic images taken under certain conditions and having specific characteristics. Raw thermographic images acquired under real-world conditions may not be immediately suitable for the non-invasive intoxication assessment methods disclosed herein. Environmental factors, such as heat radiating elements, extreme weather conditions, foreign objects obstructing thermographic image acquisition, and multiple individuals within an image frame, may affect the thermographic image quality as it relates to the non-invasive methods.

Further, even where these environmental factors do not affect the ability of the non-invasive methods to accurately assess intoxication statuses or levels, they may produce additional or erroneous information within a thermographic image that requires additional computational resources to process. In applications where time, computational resources and power sources are constrained, this issue may be magnified, potentially limiting possible real-world applications. For example respecting time-constrained applications, breathalyzer applications may require real-time acquisition of results, and significant delays may be unacceptable. For example respecting computational resource and power source constrained applications, portable devices having limited processor power and battery sources significantly benefit from operations that are more processor and energy efficient.

In embodiments disclosed herein, pre-processing of thermographic images is used to modify raw thermographic images to provide the non-invasive intoxication assessment methods with a pre-processed thermographic image representing data relating to an “ideal face”. Specifically, the “ideal face” comprises facial information relating to an individual being evaluated required for the non-invasive intoxication assessment methods while excluding non-essential or spurious information and reducing the amount of raw data being processed.

Where a raw thermographic image further comprises facial information relating to other people in addition to the individual being assessed, in embodiment disclosed herein, pre-processing identifies the individual being evaluated and excludes the other data prior to providing it to the non-invasive intoxication assessment methods. By removing facial information relating to other individuals, potential erroneous results and unnecessary computations are eliminated, resulting in a more accurate and efficient overall process.

Similarly, where pre-processing identifies and isolates facial information relating to the individual being evaluated, thermographic information relating to non-relevant environmental elements are removed, similarly resulting in a more accurate and efficient overall process.

In embodiments disclosed herein, pre-processing comprises converting thermographic images from a multi-channel format into a single-channel, greyscale format. Examples of multi-channel formats include: red, green and blue (RGB); cyan, magenta, yellow and key (CMYK), Lab Color (LAB); and indexed color. Converting from a multi-channel format to a single-channel format reduces the amount of data in a thermographic image. As the non-invasive intoxication assessment methods effectively operate using greyscale thermographic images, multi-channel format images are not required, therefore the conversion and use of single-channel images results in more efficient processing, storage and communication.

In embodiments disclosed herein, pre-processing comprises scaling, reducing, compressing or the like to reduce the size of a thermographic image. An optimal range of image size or resolution is determined by the particular application, systems (including neural network libraries), and the non-invasive intoxication assessment method used. For example, embodiments of the non-invasive intoxication assessment method requiring a particular number of data points on a facial region or a eye region will determine the range of image size or resolution required. In embodiments disclosed herein, image sizes and resolutions being a multiple of 32 are used as this permits interpolations, such that data isn't lost when images are reduced in size. Minimum image sizes and resolutions are similarly dependent on the particular application and non-invasive intoxication assessment method requirements, as well as limits resulting from the Nyquist-Shannon sampling theorem. As an example, thermographic images commonly have an image size of 800×400 pixels and images as small as 320 by 160 pixels is effectively used by non-invasive intoxication assessment methods.

In embodiments disclosed herein, pre-processing comprises normalizing pixels in a thermographic image to human body temperature. As the non-invasive methods process data relating to body temperature of a facial region of an individual, pixels representing temperatures outside a temperature range can be excluded, reducing both image size and computational/storage/communication requirements. Ranges can be selected based on accuracy requirements as well as computational/storage/communication constraints to preserve relevant human physiological temperature data. Average normal human body temperature is 37° C. (98.6° F.). For example, and without limitation, a temperature range of 37° C.±25° C. (i.e. 12° C. to 52° C.) may be used. In an embodiment, the temperature range may be 37° C.±20° C. (i.e. 17° C. to 47° C.). In an embodiment, the temperature range may be 37° C.±5° C. (i.e. 32° C. to 42° C.).

In embodiments disclosed herein, pre-processing comprises one or more computational methods including convolutional neural networks (CNN), such as YOLOv5, or Haar Cascade classifiers. Use of a particular computational method depends on suitability for a particular application. For example, use of a CNN may be preferred where computational resources are more abundant. Conversely, for example, use of a Haar Cascade may be more suitable for portable devices, where computational resources are limited.

In embodiments disclosed herein, pre-processing comprises identification of one or more obstructions in a facial region of an individual affecting the ability of the non-invasive method to assess the intoxication status or level. Examples of obstructions include a beard, a mask, a moustache, a hat, a pair of glasses, a pair of sunglasses, a neck brace, an eye patch, a medical dressing, a turtle neck shirt, a tattoo, a pair of headphones, and a pair of car muffs. In embodiments disclosed herein, a method or system may reject a thermographic and/or alert a user or operator than an obstruction has been identified. In embodiments disclosed herein, identification of an obstruction may also result in an adjustment to an intoxication status or level assessment or result in a notation.

### Methods of Non-Invasive Intoxication Detection

In an embodiment, the present disclosure relates to a non-invasive method for assessing the intoxication status or level of an individual, the method comprising: receiving a thermographic image comprising a face or facial features of the individual; performing pre-processing of the thermographic image to provide a pre-processed image; identifying a face portion comprising the face or facial features in the pre-processed image; and analyzing the face portion using an intoxication assessment method to assess the intoxication status or level.

As used herein, by “non-invasive” it is meant that the methods can operate by little to no contact with an individual, and that the methods do not require obtaining a biological sample from the individual (e.g., tissues, blood, urine, saliva, breath, etc.). In an embodiment, the non-invasive intoxication assessment methods herein are contactless, i.e. there is no contact with the individual in performing the methods.

As used herein, by “assessing the intoxication status” it is meant to refer to an assessment of whether an individual falls within a particular category of intoxication, for example of being non-intoxicated (e.g. sober), impaired or intoxicated (e.g. drunk). Each status may be based on any number of criteria, including for example when the intoxicant is alcohol, a defined blood alcohol level such as for example set by a governmental or private body.

As used herein, by “assessing the intoxication level” it is meant to refer to the ability of the disclosed methods to characterize the intoxication state of an individual to a corresponding measure of intoxication, such as for example a blood alcohol level. For example, embodiments of the disclosed methods where the intoxicant is alcohol can predict the precise level of sobriety or drunkenness to a corresponding range or even an approximate blood alcohol level. Thus, rather than broader categories of intoxication state (e.g. non-intoxicated, impaired, or intoxicated), the methods in some embodiments may predict the actual level of intoxication to a specific value or range.

As used herein, the term “intoxication” is intended to refer a state of being intoxicated or impaired by any particular substance, and in particular those substances that are capable of imparting thermal characteristics or signatures within or on the face or a facial feature of an individual. In an embodiment, the intoxication is by inebriants (e.g. alcohol, chloroform, ether, benzene, and other solvents and volatile chemicals), a recreational drug, a pharmaceutical drug (e.g. over-the-counter or prescription), or any combination thereof. In an embodiment, the intoxication is by alcohol. In an embodiment, the intoxication is by a drug. In an embodiment, the drug is one or more drugs selected from narcotics, depressants, stimulants, hallucinogenics, hypnotics, and steroids (e.g. anabolic steroids). In a particular embodiment, the intoxication is by alcohol, opioids, benzodiazepines, cannabinoids (e.g. derived from cannabis and/or synthetically produced), barbiturates, or any combination thereof. In an embodiment, the intoxication is by an opioid. In an embodiment, the intoxication is by methadone. In an embodiment, the intoxication is by a psychedelic. In an embodiment, the intoxication is by methamphetamine.

As above, in an embodiment the intoxication is by alcohol. As an example, a non-intoxicated status (e.g. sober status) might be an assessment by the methods herein to identify individuals, based on thermographic images, who should have a corresponding blood alcohol level of 0.05% or less. An impaired status may be those, based on thermographic images and the methods herein, who should have a corresponding blood alcohol level of between 0.05% and 0.08%. An intoxicated status (e.g. drunk status) may be those, based on thermographic images and the methods herein, who should have a corresponding blood alcohol level of above 0.08%. The methods herein do not require actually determining the blood alcohol level, but rather the assessment and determination of intoxication status is based on conducting analysis on thermographic images as disclosed herein.

In some embodiments of the methods disclosed herein, conducting the analysis of the face portion of thermographic images comprises one or more of: (i) determining differences between pixel values at different locations on the face in a Euclidian space or other space as transformed by a suitable process; (ii) determining temperature differences between different parts of the face; (iii) determining temperature differences between different parts of the eye; (iv) determining characteristics of blood vessels; (v) identifying and/or characterizing isothermal regions; (vi) determining characteristics of a neural network; (vii) employing Markov chains or Bayesian networks; (viii) identifying local difference patterns (LDPs); and (ix) employing a feature fusion analysis.

In some embodiments, the methods of the present disclosure involve conducting any one, two, three, four, five, six, seven, or eight of (i)-(ix). In some embodiments, additional methods are used to confirm results of other methods. In some embodiments, the methods of the present disclosure involve conducting at least three of (i)-(ix). In some embodiments, the methods of the present disclosure involve conducting all of (i)-(ix). In select embodiments, the any one, two, three, four, five, six, seven, or eight of (i)-(ix) are performed in a predefined order. In some embodiments, all of (i)-(ix) are performed in a predefined order. By “predefined order”, it is intended to mean a sequential order. However, this does not exclude the possibility that some analyses are overlapping in part or in whole.

In an embodiment, the methods of the present disclosure involve conducting the analysis in the order of (i), then (ii), then (iii), then (iv), then (v), then (vi), then (vii), then (viii), and then (ix), whereby the conducting of any of (i)-(ix) may overlap in part.

In some embodiments, conducting two, three, four, five, six, seven, eight, or all of (i)-(ix) reduces the incidence of false positives.

In some embodiments of the methods herein, the step of receiving a thermographic image of the face or the facial feature comprises a step of imaging the individual using an infrared camera. The imaging may involve taking any number of images, for example in sequence. The imaging may involve taking an image of any number of different regions of the face, whether by a single image or multiple different images.

In some embodiments, the methods herein may involve one or more configuration steps using weightings that emerge from training based on known data sets. As used herein, by “known data set” it is intended to refer to a data set that is based on previously obtained images and/or other data. The known data set may be based on pre-existing data of the individual being tested, pre-existing data from different individuals, pre-existing data from other sources, or any combination thereof. In an embodiment, the known data set is based solely on data from individuals that are not the tested individual. In an embodiment, the known data set evolves as more and more thermographic images are accumulated during usage or the methods or device disclosed herein. This allows a method using a known data set to adapt or attune itself during usage.

In some embodiments, the methods herein are capable of providing the assessment of intoxication status or level independent of a priori data for the individual. In an embodiment, this may be based on usage of the known data set. By “independent of a priori data”, it is intended to mean without pre-existing information relating to the subject being tested, such as for example a “before” image acquired prior to the individual taking any of the intoxicant (e.g. for alcohol when the individual is sober) and/or an “after” image acquired subsequent to the individual taking any of the intoxicant (e.g. for alcohol when the individual is drunk). In an embodiment, the methods herein are capable of providing the assessment of intoxication status or level independent of a non-intoxicated (e.g. sober state) thermographic image of the individual.

In some embodiments, the methods herein involve a step of comparing data obtained from the one or more thermographic images to a previously collected data set. The previously collected data set may for example be the known data set. In some embodiments, the previously collected data set comprises thermographic imaging data for one or more states or levels of intoxication or for absolute non-intoxication (e.g. sobriety). In other embodiments, the previously collected data set may be thermographic images from the tested individual at an earlier point in time. For example, the earlier point in time may be a matter of seconds, minutes or hours prior to the current thermographic images. In some embodiments, the previously collected data set is 5 seconds, 10 seconds, 30 seconds, 1 minutes, 15 minutes, 30 minutes, 1 hour, 2 hours, 3 hours, 4 hours, 5 hours, 6 hours, 7 hours, 8 hours, 9 hours, 10 hours, 11 hours, 12 hours, 18 hours, 24 hours, 2 days, 3 days, 4 days, 5 days, 6 days, 7 days, or more prior to the current thermographic images. Comparing the previously collected data set to the current data set may provide information on thermographic trends useful in assessing intoxication status or level.

### Determining Differences Between Pixel Values at Different Locations on the Face in a Euclidian Space or Other Space as Transformed by a Suitable Process

In an embodiment of the methods disclosed herein, the step of analyzing the face portion using an intoxication assessment method may comprise determining differences between pixel values at different locations on the face in a Euclidian space or other space as transformed by a suitable process. An exemplary description of such analysis follows, which is exemplary and may be altered or supplemented as appreciated by the skilled person taking into account the disclosure of the present application as a whole.

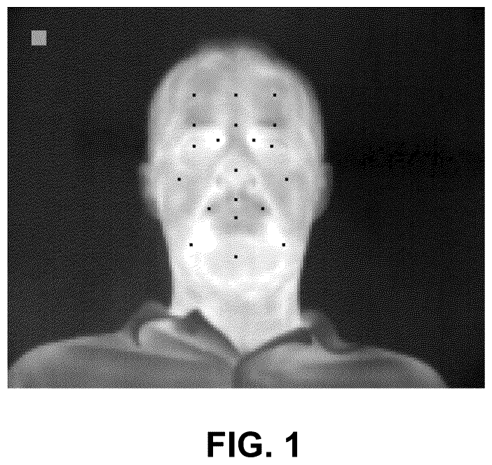

A simple feature vector was formed for assessment of intoxicated individuals by taking the pixel values of 20 different points on the face of each person (see FIG. 1). Therefore, each facial image corresponds to a 20-dimensional feature vector.

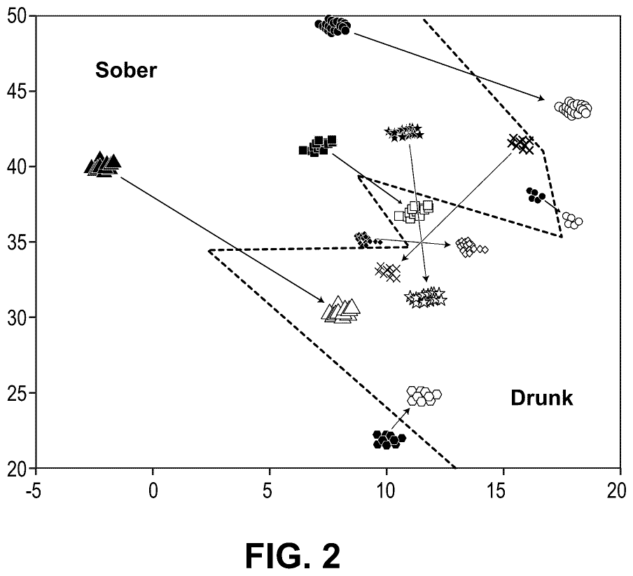

Since a set of 50 images was acquired for the same individual, a cluster of 50 points in the 20-dimensional space was formed. It was found that the cluster which corresponds to the same person moves in the feature space as the person consumes alcohol. It was also found that the clusters of different persons move towards the same direction with alcohol consumption. This is shown by means of a dimensionality reduction procedure working in the 2-dimensions, since the solution of the generalized eigenvalue problem has given only 2-eigenvalues with significant value. Bringing all clusters in the 2-dimensional space, it is evident that the clusters move in almost the same directions as the person consumes alcohol to form so-called “sober” and “drunk” regions of the feature space (FIG. 2).

The feature space dimensionality reduction and its separability into “sober” and “drunk” regions was examined by means of the Fisher Linear Discriminant (FLD) procedure. FLD is a general procedure taking into consideration that the between the clusters scatter matrix (S_{B}) and the within each cluster scatter matrix (S_{W}) have an opposite effect. To achieve that, the projection by means of a linear transformation W in a new space is required. The vectors Wᵢ of W are the new directions where (each image-vector) x will be projected. The goal for W is that in the transformed space the function J is maximized:

$$J ( w ) = \frac{{w}^{t} {S}_{B} w}{{w}^{t} {S}_{W} w}$$

The transformation vectors w that maximize the function J(w) are obtained from the solution of the generalized eigenvalue problem:

S_{B}Wᵢ=λᵢS_{W}Wᵢ

This solution provides the matrix W of eigenvectors Wᵢ, which constitute the directions in the new space, on which to project the original image vectors xᵢ. Simultaneously, this gives the eigenvalues which correspond to each of the above eigenvector. The eigenvalues express the importance of each direction-eigenvector in the feature space. The larger the eigenvalue, the better the separability of the clusters obtained towards the corresponding eigenvector.

The sum of these two largest eigenvalues over the sum of all eigenvalues gives the quality of cluster separability in the reduced (2D) feature space. In this experiment, this ratio was found equal 70%. Based on FIG. 2, it can be decided whether an unknown person is intoxicated or not, from the position of the corresponding cluster on this space, hereafter the “drunk space”.

### Determining Temperature Differences Between Different Parts of the Face

In an embodiment of the methods disclosed herein, the step of analyzing the face portion using an intoxication assessment method may comprise determining temperature differences between different parts of the face. An exemplary description of such analysis follows, which is exemplary and may be altered or supplemented as appreciated by the skilled person taking into account the disclosure of the present application as a whole.

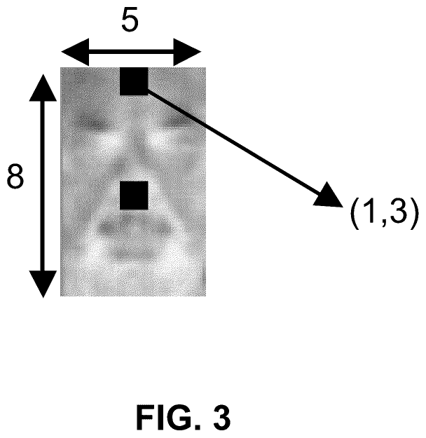

The thermal differences between various locations on the face were examined. It was determined that some regions of the face become hotter than others when consuming alcohol. According to the experimental procedure herein, the face of each person was partitioned into a matrix of 8×5 squared regions. Each region (i, j) is of 10×10 pixels and is located on the same position of the face for every image (see FIG. 3). The identification approach was based on monitoring the temperature difference between all possible pairs of squared regions as the person consumes alcohol. It was evident that the thermal difference between specific regions on the face increased for the intoxicated person.

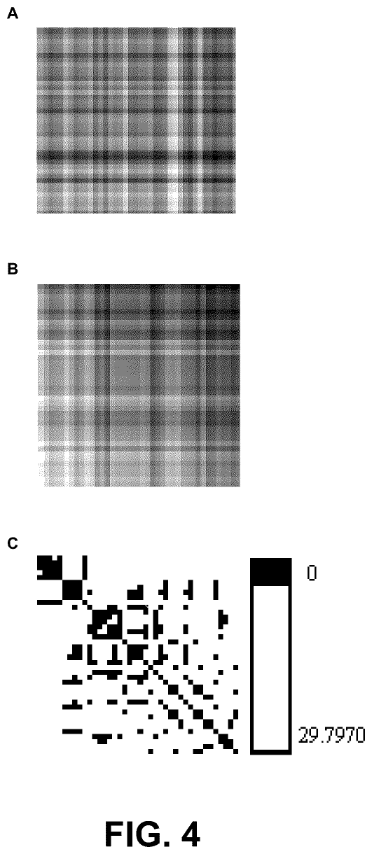

In FIG. 3, two locations are demonstrated on the face of an intoxication (i.e. drunk) person that present thermal difference. The temperature of the nose was increased compared to that of the forehead. The nose was found to become hotter than the forehead for an intoxicated person while the temperature of the nose and forehead was almost the same for a non-intoxicated (e.g. sober) person. Since the whole identification procedure was based on the thermal difference matrices, and the localizations of their maximum changed, difference matrices are present as a grey-scale 40×40 image in FIG. 4. The first matrix (FIG. 4A) corresponds to a non-intoxicated (e.g. sober) person. The second matrix (FIG. 4B) corresponds to an intoxicated (e.g. drunk) person, while their difference is presented as a third matrix (FIG. 4C) where the maximum changes can be localized. The maximum variation of the difference matrices in FIG. 4A and FIG. 4B are represented with the white pixels in the matrix of FIG. 4C. The coordinates (i, j) of these white pixels show those pairs of squared regions on the face that have maximum thermal change.

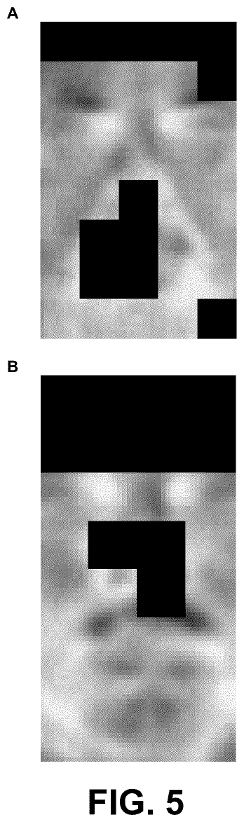

In FIG. 5, the regions that exhibit the largest change in thermal differences after alcohol consumption are demonstrated for two different people. These regions were indicated by the corresponding difference matrices as the one in FIG. 4C, as being candidates for revealing an intoxicated person in a potential alcohol test.

An individual will be identified as intoxicated if the nose is hotter than the forehead.

### Determining Temperature Differences Between Different Parts of the Eye

In an embodiment of the methods disclosed herein, the step of analyzing the face portion using an intoxication assessment method may comprise determining temperature differences between different parts of the eye. An exemplary description of such analysis follows, which is exemplary and may be altered or supplemented as appreciated by the skilled person taking into account the disclosure of the present application as a whole.

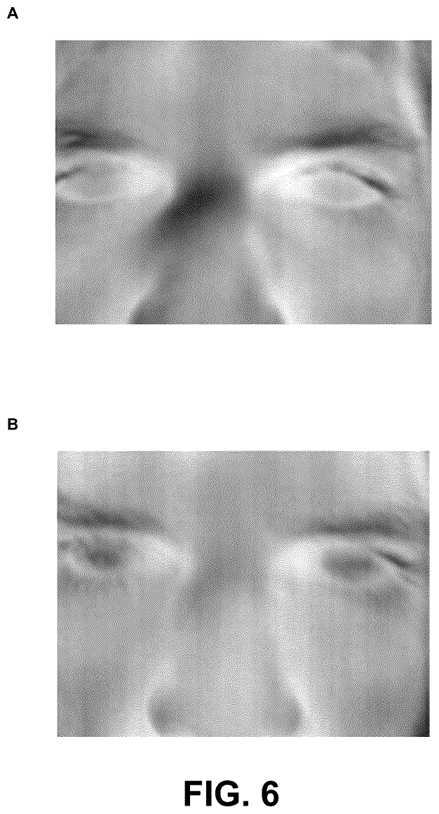

It was observed that the temperature difference between the sclera and the iris is zero or very near zero for the non-intoxicated person (FIG. 6A) and increases when an individual consumes alcohol (FIG. 6B). This was observed by the denser blood vessel network of the sclera. Histogram modification algorithms were employed to show off the gray level difference between the sclera and the iris for intoxicated persons. Such algorithms are histogram clipping, level slicing and gamma-correction. The discrimination capability of the procedure was verified using the Student t-test. It was found a confidence of over 99% in intoxicated person discrimination.

Accordingly, a thermal (infrared) imaging system was capable of capturing the thermal signature of the eyes of a person and provide an assessment as to whether a person has consumed alcohol or not. Since, for the non-intoxicated (i.e. sober) person the sclera and the iris are of the same gray level (temperature), only the infrared image of the eye of the intoxication (i.e. drunk) person is necessary for inspection for an assessment of intoxication status.

### Determining Characteristics of Blood Vessels

In an embodiment of the methods disclosed herein, the step of analyzing the face portion using an intoxication assessment method may comprise determining characteristics of blood vessels. An exemplary description of such analysis follows, which is exemplary and may be altered or supplemented as appreciated by the skilled person taking into account the disclosure of the present application as a whole.

The activity of the facial blood vessels of non-intoxicated and intoxicated people comes into sight when nonlinear anisotropic diffusion and top-hat transformation are applied to enhance and isolate the vessels from the rest information on the face. For an intoxicated person, vessels around nose and eyes as well as on the forehead become more active whereas for a person who is non-intoxicated the vessels' activity is smoother (more uniform) all over the facial thermal image. Accordingly, intoxication status and/or level can be ascertained by only using the thermal infrared image of an individual. The Student's t-test was employed to assess the degree of confidence in separating the thermal images corresponding to non-intoxicated (e.g. sober) and intoxicated (e.g. drunk) people.

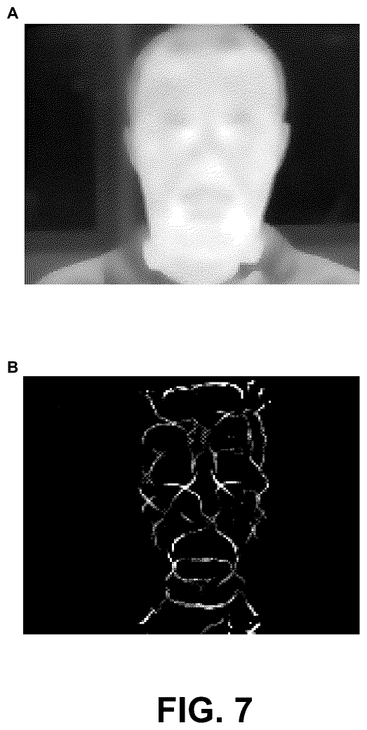

Vessels were separated and isolated from the rest of the information on the face by applying morphology on the diffused image while top-hat transformation was applied next. Accordingly, the original image was first opened and then, the opened image was subtracted from the original image. Thus, bright (hot) features like vessels were be isolated. FIG. 7A depicts the result of applying anisotropic diffusion on the image of an intoxicated person while FIG. 7B shows the corresponding vessels extracted using top-hat transformation.

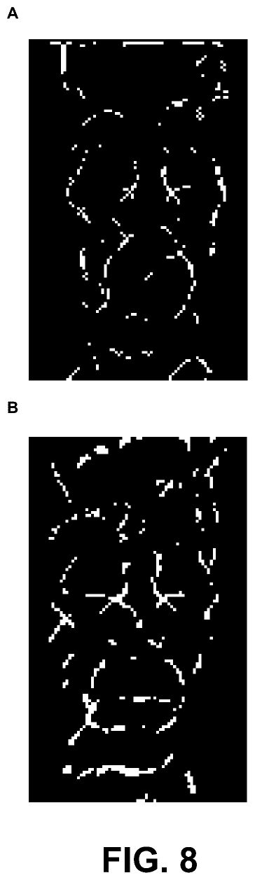

The image of the intoxicated person was registered with respect to that of the non-intoxicated in order the two images can be easily compared (FIG. 8). A piecewise linear transformation was used for this purpose. As corresponding points to apply the piecewise linear transformations were selected the intersections of the vessels. The results are similar, giving for intoxicated persons more active and bright vessels around the mouth and the nose. Furthermore, bright and distinguishable vessels were found in the forehead as well. The most prominent features used to discriminate the non-intoxicated from the intoxicated images were the active (bright) pixels. In all cases, the number of bright pixels in the faces of the intoxicated persons was larger than that on the faces of the non-intoxicated (i.e. sober) persons. Brighter vessels are a clear evidence to predict alcohol consumption and proceed to further checkup and inspection of the person.

A detailed statistical analysis procedure was employed based on the number of bright pixels to establish the existence of an intoxicated state.

### Identifying and/or Characterizing Isothermal Regions

In an embodiment of the methods disclosed herein, the step of analyzing the face portion using an intoxication assessment method may comprise identifying and/or characterizing isothermal regions. An exemplary description of such analysis follows, which is exemplary and may be altered or supplemented as appreciated by the skilled person taking into account the disclosure of the present application as a whole.

The isothermal regions on the face of a person change shape and size with alcohol consumption. Intoxication identification can be carried out based only on the thermal signatures of an intoxicated person, while the signature of the corresponding non-intoxicated person is not needed. A morphological feature vector called pattern spectrum was employed as an isotherm shape descriptor and Support Vector Machines (SVMs) were employed as classifiers.

Two different methods for isothermal region determination based on the histogram of the images was applied. Specifically: (1) The histogram range was divided into equal in width segments, and (2) Arbitrary determination of each isotherm based on the minima of the histogram.

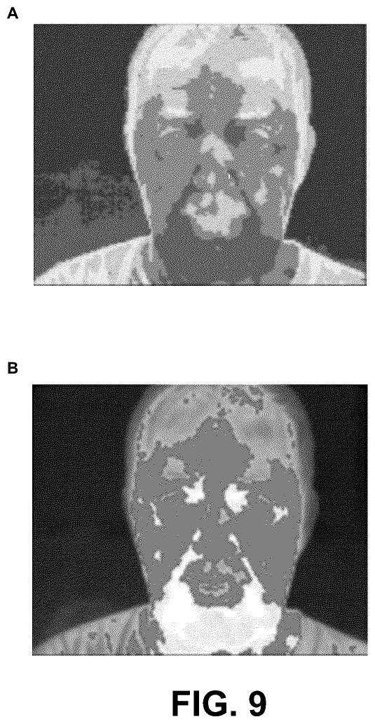

FIG. 9 illustrates one example for each method. Anisotropic diffusion was used to obtain smoother isothermal regions while morphological features are used in the SVMs for intoxication identification.

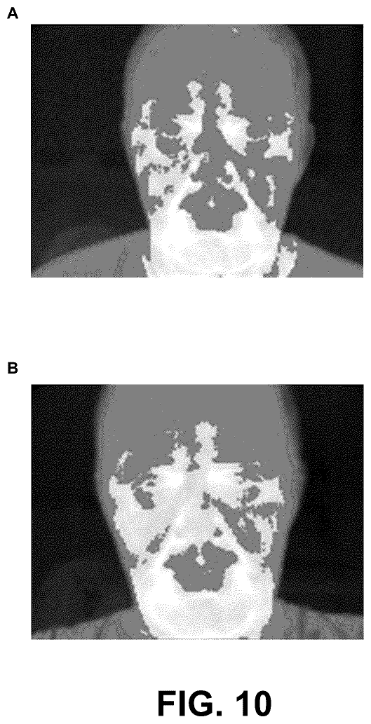

The arbitrary defined regions are schematically depicted in FIG. 10A-10B. It was evident that the regions became larger as the person consumes alcohol. With this approach features were derived for identifying a person as being intoxicated without the need of information from the non-intoxicated person. It was found that the region of the forehead for a non-intoxicated (e.g. sober) person lies in the same isothermal range with other regions of the face. In contrast, for an intoxicated person the region of the forehead is isolated and lies in its own isothermal region. Consequently, an isothermally isolated forehead corresponds to an intoxicated state.

SVMs map the clusters of two categories so that a clear wide gap separates them. This gap prevents new incoming samples to be incorrectly classified. By employing the so-called ‘kernels’, SVMs achieve non-linear classification by mapping the samples into a higher dimensionality feature space.

Pattern spectrum and impulsive spectral components were employed as features from these isothermal regions. The performance of various types of SVMs were tested, namely Linear, Precomputed, Polynomial, Radial basis and Sigmoidal Kernels. The combination of these types of SVMs, with two different morphological feature vectors, and the three types of isotherms with or without diffusion, gave 60 different cases for testing intoxication by means of isotherms.

The simple spectrum without performing diffusion achieves the largest success which reaches 86%. Pre-computed and Linear Kernel types were employed in this case. Consequently, an interesting result was obtained in that an intoxicated person was identified since the isothermal regions, in which the forehead lies, contain no other region of the face. Assessment of intoxicated state was obtained without comparison with the infrared image of the non-intoxicated person. The method is non-invasive and provides a fast means for intoxication detection.

### Determining Characteristics of a Neural Network

In an embodiment of the methods disclosed herein, the step of analyzing the face portion using an intoxication assessment method may comprise determining characteristics of a neural network. An exemplary description of such analysis follows, which is exemplary and may be altered or supplemented as appreciated by the skilled person taking into account the disclosure of the present application as a whole.

The neural networks were employed as a black box to discriminate intoxication by means of the values of simple pixels from the thermal images of the persons' face. The neural networks were used by means of two different approaches.

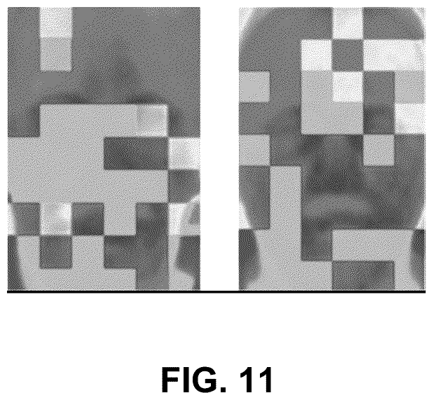

According to the first approach, a different neural structure was used from location to location on the thermal image of the face and includes identifying a high correlation area comprising a high correlation between vectors extracted from the test subject and those deduced during training. Successful identification of correlation by a neural network to a minimum value in a specific location means that this face location is suitable for assessment of intoxication status or level, and for intoxication identification. For demonstration purposes, a region for which high correlation of the network was observed is given at darker areas in FIG. 11. High correlation was observed mainly on the forehead, the nose, and the mouth. Thus, these locations of the face of a person are the most suitable to be employed for intoxication discrimination and assessment of intoxication status or level.

According to the second approach, a single neural structure was trained with data from the thermal images of the whole face of a person (non-intoxicated/sober and intoxicated/drunk) and its capability to operate with high classification success to other persons was tested. The whole face of each specific person was examined as a single area of 5000 pixels. The object of this approach was to discriminate between the non-intoxicated and the intoxicated image of a person using a specific neural structure which has been trained with information coming from the images of another person.

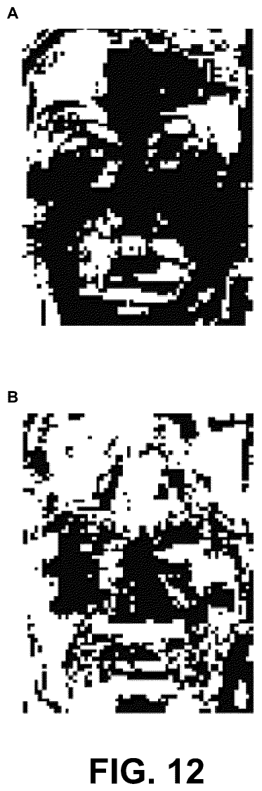

A neural structure of 3 layers with 49 neurons in the first layer, 49 neurons in the hidden layer and 1 neuron in the output layer was trained with the above data. The same trained network was tested with the same data and resulted in satisfactory performance. When the output was closer to zero, the pixel was declared to belong to a non-intoxicated (i.e. sober) person (black), otherwise (closer to one) it was declared to represent an intoxicated (i.e. drunk) person (white). Simultaneously, the performance of the network was tested on the images of another person (both when sober and after having consumed alcohol). The results were satisfactory since, as shown in FIG. 12, the image of the non-intoxicated person is mostly black while the image of the intoxicated person is mostly white. Consequently, no a priori data records of the inspected persons (e.g. sober state data) was needed for assessment of intoxication status.

### Employing Markov Chains or Bayesian Networks

In an embodiment of the methods disclosed herein, the step of analyzing the face portion using an intoxication assessment method may comprise employing Markov chains or Bayesian networks. An exemplary description of such analysis follows, which is exemplary and may be altered or supplemented as appreciated by the skilled person taking into account the disclosure of the present application as a whole.

Markov chains were used to model the statistical behavior of the pixels on the thermal image of the forehead of a person to detect intoxication. Intoxication affects blood vessel activity, which has a significant effect on the corresponding pixels statistics. The pixels of the forehead images were quantized to 32 gray levels so that Markov chain models are structured using 32 states. The feature vectors used were the eigenvalues obtained from the first order transition matrices of the Markov chain models. Since a frame sequence of 50 views was acquired for each person, a cluster of 50 vectors was formed in the 32-Dimensional feature space.

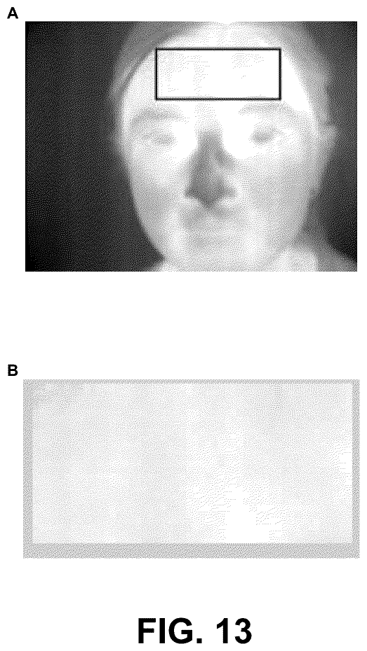

Measurements applying Markov models were carried out on the region of the forehead as shown in FIG. 13A-13B. This forehead region was configured to have 25×50 pixels size for each specific participant in the experiment. The pixel values in this region of the forehead were quantized, separately for a non-intoxicated (sober) and an intoxicated person, into a histogram of 32 equal-spaced bins. Thus, for each person two different transition matrices were created, one for the non-intoxicated person image and another for the image of the intoxicated counterpart.

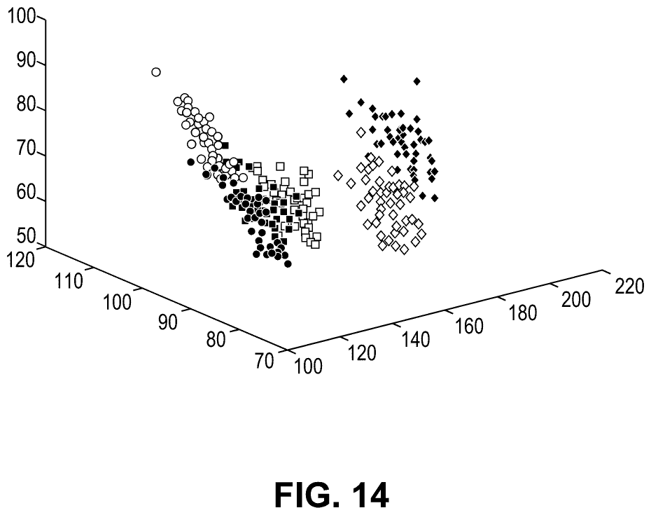

The feature space was investigated using FLD Analysis as far as clusters separability is concerned (non-intoxicated or intoxicated persons). Trivial projection from the 32-dimensions of the original space into 3 dimensions (FIG. 14), reveals that the selected features are useful candidates for assessment of intoxication status and level. To elaborate on non-intoxicated and intoxicated cluster formation, their representation in the existing feature space was demonstrated by plotting the clusters of three persons in three of the 32 existing dimensions. Accordingly, the clusters are well separated as is obvious from FIG. 14 (Person 1=circles; Person 2=Squares; Person 3=diamonds).

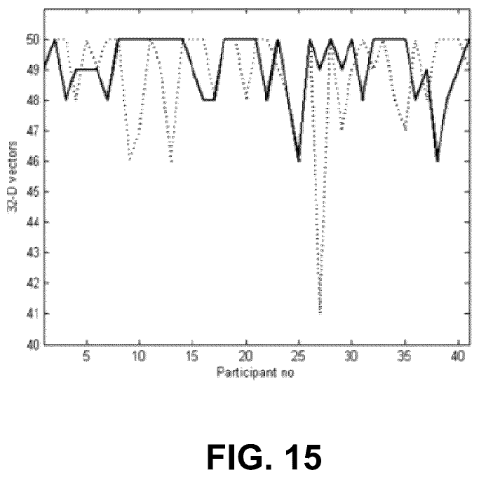

The capability of a simple feed forward Neural Network to separate the clusters belonging to non-intoxicated (e.g. sober) persons from those corresponding to intoxicated persons was investigated. A simple three-layer neural structure has a 98% vector separability success and a 100% cluster separability if the majority voting is considered (FIG. 15). Furthermore, the classification problem is addressed by excluding from the training procedure some of the persons, and using them in the testing phase. The obtained neural structure tested with the features of the persons in which it was not trained presented high reproducibility and success in assessment of intoxication status if the majority voting is considered.

### Identifying Local Difference Patterns (LDPs)

In an embodiment of the methods disclosed herein, the step of analyzing the face portion using an intoxication assessment method may comprise identifying local difference patterns (LDPs). An exemplary description of such analysis follows, which is exemplary and may be altered or supplemented as appreciated by the skilled person taking into account the disclosure of the present application as a whole.

The region of the forehead of the face of the non-intoxicated and the corresponding intoxicated person were used to test if the employed local difference patterns constitute discriminative features. The local difference patterns employed ignore orientation of the pixels distribution and give emphasis on the first and second norms of the differences as well as the ordered values of the pixels in the employed kernels.

Small kernels 3×3 and 5×5, called LDPs were used to extract a means of local variation of the pixels into the specific kernel. After that, the values obtained were statistically described using histogram features. The forehead from non-intoxicated and intoxicated persons gave different distributions of these statistics of the pixels and thus they were easily discriminable.

For the case that the LDPs are based on the sample norms, the window employed was of size 3×3 and 5×5. In the first case of the 3×3 moving window using the first order norms the following relation was applied:

$${z}_{1} = {\sum }_{i = 1}^{8} \vert {y}_{i} - x \vert$$

For this specific 3×3 kernel a value y was extracted using the summation of the absolute difference of each pixel around the central one. In a similar way another statistic was formed based on the second order norm as follows:

$${z}_{2} = {\sum }_{i = 1}^{8} {( {y}_{i} - x )}^{2}$$

In case of a 5×5 moving window, a statistic based on the first order norm was formed as follows:

$${z}_{3} = {\sum }_{i = 1}^{24} \vert {y}_{i} - x \vert$$

while the statistic based on the second norm was evaluated as follows:

$${z}_{4} = {\sum }_{i = 1}^{24} {( {y}_{i} - x )}^{2}$$

For the case that the LDPs are based on the ordered samples of the moving window, the samples in the window were firstly seen as a vector and subsequently were sorted in ascending order. Then the difference statistics were created based on the difference of the ordered samples in the following way:

$${z}_{5} = {x}_{( 9 )} - {x}_{( 1 )} \\ or \\ {z}_{6} = {x}_{( 8 )} - {x}_{( 2 )}$$

Finally, for the case of 5×5 moving window, the relative statistics were formed using the difference of ordered sample or difference of the average value of them as described by the following equations:

$${z}_{7} = {x}_{( 23 )} - {x}_{( 3 )} \\ {z}_{8} = \frac{{x}_{( 25 )} + {x}_{( 22 )} + {x}_{( 21 )}}{3} - \frac{{x}_{( 5 )} + {x}_{( 4 )} + {x}_{( 3 )}}{3}$$

The statistics obtained according to the above were independent of the orientation of the kernel and depend on the pixel variability. The first and second norm give special attention to all values with the second norm weighting more the larger ones. The LPDs based on ordered statistics and especially z₆ and z₈ are more robust to the presence of outliers (spiky noise).

Each of the statistics z, as shown above, are evaluated all over the forehead of each person both in the case of non-intoxicated (sober) and intoxicated individuals. Accordingly, for each non-intoxicated person, and each statistic, a 16-bin histogram was created. Similar histograms are evaluated for the intoxicated persons as well. These were the features used for intoxication assessment and identification of intoxication status and level. Consequently, the analysis was performed on the 16-dimensional feature space. The histograms were properly normalized so that for the same person and statistic both histograms of the non-intoxicated and intoxicated image were divided by their common maximum value so that the 16-dimensional feature-histograms are comparable.

The histograms corresponding to intoxicated persons presented higher pixel variability. This was expected since for the intoxicated person the temperature on the face presents higher variability. Consequently, this was the basis to discriminate non-intoxicated from intoxicated persons. One can assess and identify intoxication if the cumulative values above a specific threshold constitute the majority of the values. If the majority of the values are below this threshold, then the person is not intoxicated. The intoxication status was successfully detected 83% of the time. Accordingly, a significant performance was obtained with a very simple pattern. This performance can be considered as the classification success of the procedure when statistic z₁ is employed, with an equivalent classification error equal to 17%. Additionally, the selected LPD feature can be considered very simple and easily applicable. An important advantage is that the infrared image of the non-intoxicated (e.g. sober) person is not needed for comparison to assess intoxication status or level of any given individual.

### Employing a Feature Fusion Analysis

In an embodiment of the methods disclosed herein, the step of analyzing the face portion using an intoxication assessment method may comprise employing a feature fusion analysis. An exemplary description of such analysis follows, which is exemplary and may be altered or supplemented as appreciated by the skilled person taking into account the disclosure of the present application as a whole.

Dissimilar features coming from the thermal images of the face were fused by means of neural networks. The features had been derived using different image analysis techniques and thus they convey dissimilar information, which had to be transferred onto the same framework and fused to result in an approach and assessment with improved reliability.

The first simple feature vector for identification of an intoxicated state was obtained by simply taking the pixel values of 20 different points on the face of each person. The generalized eigenvalue problem was solved and in the resulting 2-dimensional feature space the sum of the two largest eigenvalues over the sum of all eigenvalues gave the quality of cluster separability in the reduced feature space. Based on the FLD analysis, the created 2-D feature vector was used in conjunction (fusion) with the features obtained from the persons eyes in order to create the final feature vector. Hereafter, the following notation for the above 2-D feature was used:

$${x}_{a} = [ \begin{matrix}{x}_{a 1} \\ {x}_{a 2}\end{matrix} ]$$

Temperature distribution on the eyes of non-intoxicated and intoxicated persons was the second feature vector used in the tested feature fusion procedure. In most cases the sclera is brighter than the iris for intoxicated persons, while histogram modification algorithms can display the grey level difference between them for intoxicated persons. The discrimination capability of the procedure was verified using the Student t-test and a confidence of over 99% in assessment of intoxicated state was achieved. Accordingly, two different discrimination features were derived. The first one x_{b1}, is the ratio of the mean value of the pixels inside the sclera to the mean value of the pixels inside the iris.

This procedure was performed on the left eye of each participant, both when the participant was in a non-intoxicated state and after consumption of alcohol. The second feature x_{b2} corresponds to the variance of the pixels contained in the whole eye. It was observed that the variance increases in instances where the person has consumed alcohol. Consequently, a 2-D feature vector can be obtained employing features x_{b1} and x_{b2} as follows:

$${x}_{b} = [ \begin{matrix}{x}_{b 1} \\ {x}_{b 2}\end{matrix} ]$$

The fusion procedure refers to manipulating the correlation between the above features xₐ and x_{b}, as well as the importance of each of the features by weighting them with proper coefficients. The final feature vector to be transferred to the Neural Networks was as follows:

$$x = [ \begin{matrix}{x}_{a} \\ {x}_{b}\end{matrix} ] = [ \begin{matrix}{x}_{a 1} \\ {x}_{a 2} \\ {x}_{b 1} \\ {x}_{b 2}\end{matrix} ]$$

The association matrix Sₐ, which reveals weak or strong correlation between the selected features, was evaluated from the expectation:

Sₐ=E{xx^{t}}

It was found, by obtaining the association matrix (correlation coefficient matrix) that some of the correlation coefficients between the four components were quite small. Low correlation means that each feature component contains different information compared to the rest of the components. Accordingly, all this information could be exploited. This fact permits for an increased classification performance when all features are used together. In the following the correlation coefficients matrices are given for the non-intoxicated and intoxicated persons, respectively:

### Correlation Coefficients for the Sober Persons

| | | | |
|---|---|---|---|
| 1.00 | −0.09 | −0.79 | −0.74 |
| −0.09 | 1.00 | 0.25 | 0.17 |
| −0.79 | 0.25 | 1.00 | 0.71 |
| −0.74 | 0.17 | 0.71 | 1.00 |

### Correlation Coefficients for the Drunk Persons

| | | | |
|---|---|---|---|
| 1.00 | 0.67 | −0.30 | 0.22 |
| 0.67 | 1.00 | −0.11 | 0.19 |
| −0.30 | −0.11 | 1.00 | 0.75 |
| 0.22 | 0.19 | 0.75 | 1.00 |

The dissimilar features were fused using neural networks. The approach followed in the experimental procedure was to test the performance of the available features based on two criteria. The first one was how small the neural structure could be that classifies the intoxicated person correctly. The second criterion was how fast this structure can converge during the training procedure. It was found that a two-layer neural network is needed for converging to a high classification success. A network with 8 neurons in the first layer was adequate for achieving a high classification rate of 99.8%.

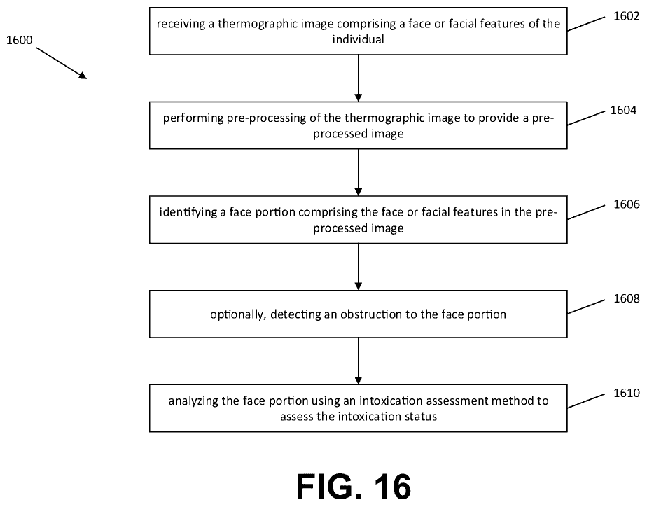

FIG. 16 is a flowchart showing the steps of a method for assessing an intoxication status of an individual 1600, according to one embodiment of the present disclosure. The function 1600 begins with receiving a thermographic image comprising a face or facial features of the individual (step 1602). At step 1604, pre-processing of the thermographic image is performed to provide a pre-processed image. At step 1606, a face portion comprising the face or facial features in the pre-processed image is identified. At step 1608, optionally, an obstruction to the face portion is detected. At step 1610, the face portion is analyzed using an intoxication assessment method to assess the intoxication status.

The aspects of the methods disclosed herein may be used alone or in combination to create a reliable means of assessing the intoxication level of an individual in a contactless manner by using a thermal image of the face.

### Systems and Devices

In certain embodiments, the methods herein take the form of a commercially available thermographic camera connected to a general purpose computation device and located in a housing approved for use in a given jurisdiction. The camera and the computation device may access encrypted digital media embodied in either a memory card or present on a remote server.

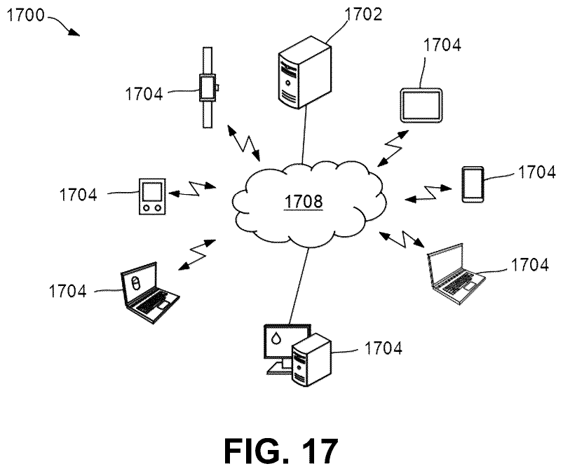

Embodiments of intoxication assessment systems disclosed herein may be implemented on a variety of computer network architectures. Referring to FIG. 17, a computer network system for intoxication assessment is shown and is generally identified using reference numeral 1700. As shown, the computer network system 1700 comprises one or more computers 1702 and a plurality of computing devices 1704 functionally interconnected by a network 1708, such as the Internet, a local area network (LAN), a wide area network (WAN), a metropolitan area network (MAN), and/or the like, via suitable wired and wireless networking connections.

The computers 1702 may be computing devices designed specifically for executing the non-invasive methods disclosed herein and/or general-purpose computing devices. The computing devices 1704 may be portable and/or non-portable computing devices such as application-specific devices such as kiosks and terminals, as well as laptop computers, tablets, smartphones, Personal Digital Assistants (PDAs), desktop computers, and/or the like. Each computing device 1704 may execute one or more client application programs which sometimes may be called “apps”.

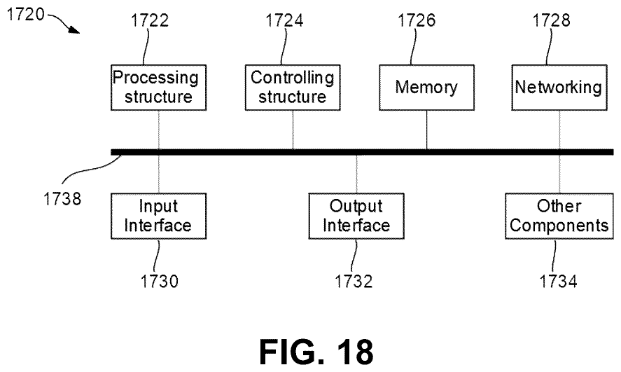

Generally, the computing devices 1702 and 1704 have a similar hardware structure such as a hardware structure 1720 shown in FIG. 18. As shown, the computing device 1702/1704 comprises a processing structure 1722, a controlling structure 1724, one or more non-transitory computer-readable memory or storage devices 1726, a network interface 1728, an input interface 1730, and an output interface 1732, functionally interconnected by a system bus 1738. The computing device 1702/1704 may also comprise other components 1734 coupled to the system bus 1738.

The processing structure 1722 may be one or more single-core or multiple-core computing processors (also called “central processing units” (CPUs)) such as INTEL® microprocessors (INTEL is a registered trademark of Intel Corp., Santa Clara, CA, USA), AMD® microprocessors (AMD is a registered trademark of Advanced Micro Devices Inc., Sunnyvale, CA, USA), ARM® microprocessors (ARM is a registered trademark of Arm Ltd., Cambridge, UK) manufactured by a variety of manufactures such as Qualcomm of San Diego, California, USA, under the ARM® architecture, or the like. When the processing structure 1722 comprises a plurality of processors, the processors thereof may collaborate via a specialized circuit such as a specialized bus or via the system bus 1738.

The processing structure 122 may also comprise one or more real-time processors, graphics processing units (GPUs), programmable logic controllers (PLCs), microcontroller units (MCUs), μ-controllers (UCs), specialized/customized processors and/or controllers using, for example, field-programmable gate array (FPGA) or application-specific integrated circuit (ASIC) technologies, and/or the like.

Generally, each processor of the processing structure 1722 comprises necessary circuitries implemented using technologies such as electrical and/or optical hardware components for executing one or more processes as the implementation purpose and/or the use case may be, to perform various tasks. In many embodiments, the one or more processes may be implemented as firmware and/or software stored in the memory 1726 and may be executed by the one or more processors of the processing structure 1722. Those skilled in the art will appreciate that, in these embodiments, the one or more processors of the processing structure 1722, are usually of no use without meaningful firmware and/or software.

The controlling structure 1724 comprises one or more controlling circuits, such as graphic controllers, input/output chipsets, and the like, for coordinating operations of various hardware components and modules of the computing device 1702/1704.

The memory 1726 comprises one or more one or more non-transitory computer-readable storage devices or media accessible by the processing structure 1722 and the controlling structure 124 for reading and/or storing instructions for the processing structure 1722 to execute, and for reading and/or storing data, including input data and data generated by the processing structure 1722 and the controlling structure 1724. The memory 1726 may be volatile and/or non-volatile, non-removable or removable memory such as RAM, ROM, EEPROM, solid-state memory, hard disks, CD, DVD, flash memory, or the like. In use, the memory 1726 is generally divided into a plurality of portions for different use purposes. For example, a portion of the memory 1726 (denoted as storage memory herein) may be used for long-term data storing, for example, for storing files or databases. Another portion of the memory 1726 may be used as the system memory for storing data during processing (denoted as working memory herein).

The system bus 1738 interconnects various components 1722 to 1734 enabling them to transmit and receive data and control signals to and from each other.

In some embodiments, the software on the computation device 1704 may comprise computer code and configurations that are designed to receive and process several images from the camera. The code for example can isolate a human face, segment it, determine sample points, take those samples and run the extracted data through a series of algorithms to perform the methods as disclosed herein. The computing device 1704, running this software and having performed its deliberations, can output a value that enumerates the assessment of the intoxication status or level of an individual. In an embodiment, these data can be expressed on a networked device, screen, matrix describer or can be used to drive LEDs, buzzers, sounds samples or any number of means for alerting an operator or automated sentry.

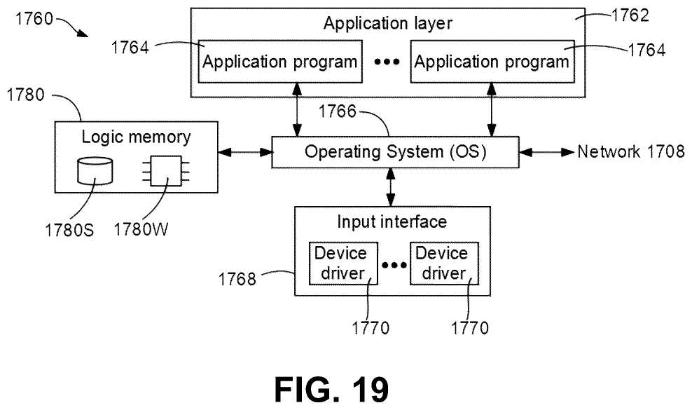

FIG. 19 shows a simplified software architecture 1760 of the computing device 1702 or 1704. The software architecture 1760 comprises an application layer 1762, an operating system 1766, a logical input/output (I/O) interface 1768, and a logical memory 1772. The application layer 1762, operating system 1766, and logical I/O interface 1768 are generally implemented as computer-executable instructions or code in the form of software programs or firmware programs stored in the logical memory 1772 which may be executed by the processing structure 1722.

Herein, a software or firmware program is a set of computer-executable instructions or code stored in one or more non-transitory computer-readable storage devices or media such as the memory 1726, and may be read and executed by the processing structure 1722 and/or other suitable components of the computing device 1702/1704 for performing one or more processes. Those skilled in the art will appreciate that a program may be implemented as either software or firmware, depending on the design purposes and requirements. Therefore, for ease of description, the terms “software” and “firmware” may be interchangeably used herein.

Herein, a process has a general meaning equivalent to that of a method, and does not necessarily correspond to the concept of computing process (which is the instance of a computer program being executed). More specifically, a process herein is a defined method implemented as software or firmware programs executable by hardware components for processing data (such as data received from users, other computing devices, other components of the computing device 1702/1704, and/or the like). A process may comprise or use one or more functions for processing data as designed. Herein, a function is a defined sub-process or sub-method for computing, calculating, or otherwise processing input data in a defined manner and generating or otherwise producing output data.

Referring back to FIG. 19, the application layer 1762 comprises one or more application programs 1764 executed by or performed by the processing structure 1722 for performing various tasks.

The operating system 1766 manages various hardware components of the computing device 1702 or 1704 via the logical I/O interface 1768, manages the logical memory 1772, and manages and supports the application programs 1764. The operating system 1766 is also in communication with other computing devices (not shown) via the network 1708 to allow the application programs 1764 to communicate with programs running on other computing devices. As those skilled in the art will appreciate, the operating system 1766 may be any suitable operating system such as MICROSOFT® WINDOWS® (MICROSOFT and WINDOWS are registered trademarks of the Microsoft Corp., Redmond, WA, USA), APPLE® OS X, APPLE® iOS (APPLE is a registered trademark of Apple Inc., Cupertino, CA, USA), UNIX, QNX, Linux, ANDROID® (ANDROID is a registered trademark of Google Inc., Mountain View, CA, USA), or the like. The computing devices 1702 and 1704 of the computer network system 1700 may all have the same operating system, or may have different operating systems.

The logical I/O interface 1768 comprises one or more device drivers 1770 for communicating with respective input and output interfaces 1730 and 1732 for receiving data therefrom and sending data thereto. Received data may be sent to the application layer 1762 for being processed by one or more application programs 1764. Data generated by the application programs 1764 may be sent to the logical I/O interface 1768 for outputting to various output devices (via the output interface 1732).

The logical memory 1772 is a logical mapping of the physical memory 1726 for facilitating the application programs 1764 to access. In this embodiment, the logical memory 1772 comprises a storage memory area that may be mapped to a non-volatile physical memory such as hard disks, solid-state disks, flash drives, and/or the like, generally for long-term data storage therein. The logical memory 1772 also comprises a working memory area that is generally mapped to high-speed, and in some implementations, volatile physical memory such as RAM, generally for application programs 1764 to temporarily store data during program execution. For example, an application program 1764 may load data from the storage memory area into the working memory area, and may store data generated during its execution into the working memory area. The application program 1764 may also store some data into the storage memory area as required or in response to a user's command.

In a computer 1702, the application layer 1762 generally comprises one or more server-side application programs 1764 which provide(s) server functions for managing network communication with computing devices 1704 and facilitating collaboration between the computer 1702 and the computing devices 1704. Herein, the term “server” may refer to a computer 1702 from a hardware point of view, or to a logical server from a software point of view, depending on the context.

As described above, the processing structure 1722 is usually of no use without meaningful firmware and/or software. Similarly, while a computer system 1700 may have the potential to perform various tasks, it cannot perform any tasks and is of no use without meaningful firmware and/or software. As will be described in more detail later, the computer system 1700 described herein, as a combination of hardware and software, generally produces tangible results tied to the physical world, wherein the tangible results such as those described herein may lead to improvements to the computer and system themselves.

For providing non-invasive methods of detecting intoxication in persons using thermal signatures as described herein, the methods described that analyze the thermographic data to determine the intoxication of the subject can either be running on the computing device 1704 itself (where the infra-red camera is housed), or alternatively, if the computing device 1704 is networked, the thermal imaging data is sent to the computer 1702 or a remote computation device (e.g. in the cloud) which determines using the same methods described herein whether the person is non-intoxicated or intoxicated, and sends back the result to the computing device 1704.

The network interface 1728 comprises one or more network modules for connecting to other computing devices or networks through the network 108 by using suitable wired or wireless communication technologies such as Ethernet, WI-FI® (WI-FI is a registered trademark of Wi-Fi Alliance, Austin, TX, USA), BLUETOOTH® (BLUETOOTH is a registered trademark of Bluetooth Sig Inc., Kirkland, WA, USA), Bluetooth Low Energy (BLE), Z-Wave, Long Range (LoRa), ZIGBEE® (ZIGBEE is a registered trademark of ZigBee Alliance Corp., San Ramon, CA, USA), wireless broadband communication technologies such as Global System for Mobile Communications (GSM), Code Division Multiple Access (CDMA), Universal Mobile Telecommunications System (UMTS), Worldwide Interoperability for Microwave Access (WiMAX), CDMA2000, Long Term Evolution (LTE), 3GPP, 5G New Radio (5G NR) and/or other 5G networks, and/or the like. In some embodiments, parallel ports, serial ports, USB connections, optical connections, or the like may also be used for connecting other computing devices or networks although they are usually considered as input/output interfaces for connecting input/output devices.

For the rejection of false positives (e.g. a person who simply has a fever), the software implements further algorithms and configurations that effectively differentiate between person who may present similar thermographic images but for different reasons.

For providing a method that works independent of a priori data on an individual, the software also implements algorithms and configurations, based on the methods described herein, that are able to identify intoxicated persons without prior knowledge of their “non-intoxicated ” state (e.g. sober). The output from the algorithms can be used to drive output devices in the manner already described. This embodiment does not however preclude the use of “before” and “after” imaging in, for example, air crew screening for additional accuracy and security. In an embodiment, the “before/after” feature can be activated or deactivated by a switch, automated configuration, or any other means.

The input interface 1730 comprises one or more input modules for one or more users to input data via, for example, touch-sensitive screens, touch-pads, keyboards, computer nice, trackballs, joysticks, microphones (including for voice commands), scanners, cameras, and/or the like. The input interface 1730 may be a physically integrated part of the computing device 1702/1704 (for example, the touch-pad of a laptop computer or the touch-sensitive screen of a tablet), or may be a device physically separated from but functionally coupled to, other components of the computing device 1702/1704 (for example, a computer mouse). The input interface 1730, in some implementation, may be integrated with a display output to form a touch-sensitive screen.

The output interface 1732 comprises one or more output modules for output data to a user. Examples of the output modules include displays (such as monitors, LCD displays, LED displays, projectors, and the like), speakers, printers, virtual reality (VR) headsets, augmented reality (AR) goggles, haptic feedback devices, and/or the like. The output interface 1732 may be a physically integrated part of the computing device 1702/1704 (for example, the display of a laptop computer or a tablet), or may be a device physically separate from but functionally coupled to other components of the computing device 1702/1704 (for example, the monitor of a desktop computer). The computing device 1702/1704 may also comprise other components 1734 such as one or more positioning modules, temperature sensors, barometers, inertial measurement units (IMUs), and/or the like.

For providing a signalling means for use by other systems, the computing device 1704 herein may comprise a signalling means to send signals to other systems. The signalling means may, for example, arise from the device's CPU. CPUs can be used to drive a wide variety of devices and given the breadth of application, any number of them may be incorporate into a device as disclosed herein. They include, for example and without limitation, (i) signals to other chips using low PCB level protocols like I2C; (ii) RS232, RS422 and RS485; (iii) driving GPIO pins commanding LEDs, buzzers, relays, serial devices or modems; (iv) driving data to storage media as described above; and (v) driving traffic to networks as described above. In some embodiments, these would be static, for example in an airport at a departure gate or at a stadium beer dispenser, while in other embodiments these may be manually applied such as a hand-held device for use by mobile law enforcement.

For providing a system of logging that will stand up to legal challenge, the measurement and returned value from the methods disclosed herein may be logged on encrypted media in real time, containing information about the algorithm and configuration versions, date and time, device ID, operator ID, ambient temperature, humidity, location and so on. For these purposes, a computing device 1704 as disclosed herein would be able to integrate a number of additional sensors that would form a precise record of then the sample was taken, where, by whom (if applicable) and under what conditions. Data retained may be available for download and analysis for the purpose of organisational statistics, research, algorithm development, and configuration improvements, as well as to form part of a record of fact suitable for legal or administrative processes. All data may be stored and returned with checksums to ensure accuracy. For those data stored on a network, blockchain technology may be used to ensure fidelity.

For providing a reliability scoring method, the algorithm of the methods disclosed herein may be capable of enumerating the probability that a person is intoxicated (e.g. drunk). The enumeration may be displayable by some means as described herein enabling deployment in settings where some tolerance can be built in. For example, law enforcement making a traffic stop may have a policy to err on the side of caution, while a device deployed at a sports stadium serving alcohol will be more permissive. The notifications thresholds can be tunable in each case, for example via onboard configuration, a connected system or in embedded implementations using DIP switches or potentiometers.

For all of the above embodiments and others, the present disclosure provides a device for assessing the intoxication status or level of an individual, the device comprising an infrared camera to provide one or more thermographic images of an individual.

The system is capable of performing the methods disclosed herein for providing the assessment of the intoxication status or level of an individual. Indeed, the methods disclosed herein are capable of being integrated and used in any number of different devices for numerous applications. For example, and without limitation, the methods can be: (i) integrated into self-service intoxicant (e.g. alcohol) dispensing kiosks to avoid over-serving an intoxicated customer; (ii) used in a stand-alone or hand-held device for use by law enforcement as a non-invasive screening tool for identifying intoxicated drivers to increase road safety; (iii) integrated into personal or company fleet vehicles or machinery to prevent an intoxicated person from operating the vehicle or machinery (e.g. heavy machinery); or (iv) used to screen employees as they enter job sites that require a low or zero threshold for intoxication (e.g. sobriety).

In essence, the methods herein have potential application in any situation for which it is desirable to have a non-invasive means of assessing a status or level of intoxicated (e.g. non-intoxicated, impaired, and/or intoxicated), and in any device for this purpose.

In many instances, existing means of intoxication assessment for alcohol include using a breathalyzer to measure breath alcohol content, or using a blood test to measure blood alcohol content. These techniques suffer from several disadvantages, some of which include their invasiveness as each method requires physical contact between the person being assessed and the apparatus that is performing the assessment and require internal sample collection from the person being assessed (in the form of giving breath or blood). Blood tests also require laboratory analysis after sample collection which is timely and costly.

Comparatively, the methods and systems disclosed herein provide a completely contactless and hygienic means of assessing intoxication status or level of an individual. This may be particularly relevant in times of a global pandemic, such as experienced with the COVID-19 pandemic. Assessment via the methods and systems disclosed herein is faster, less invasive, and will be less costly than existing methods of assessing intoxication.

In some embodiments, the system comprises a computation device capable of conducting an analysis of the one or more thermographic images to provide an assessment of the intoxication status or level of the individual.

In some embodiments, the device comprises a network communication system to transmit the one or more thermographic images to a remote computation device capable of conducting an analysis of the one or more thermographic images to provide an assessment of the intoxication status or level of the individual. The assessment result may then be transmitted back to the device to provide an indication of the intoxication status of the individual, and/or may be stored.

For the devices disclosed herein, the computation device (whether internal or remote) is capable of conducting the analysis of the one or more thermographic images by using one or more of the methods disclosed herein (e.g. (i)-(ix) as described herein). In some embodiments, in conducting the analysis of the one or more thermographic images, the computation device performs one, two, three, four, five, six, seven, eight, or all of (i)-(ix). In some embodiments, in conducting the analysis of the one or more thermographic images, the computation device performs all of (i)-(ix) in a predefined order as described elsewhere herein.

In some embodiments, the device comprises a reliable scoring method for presentation to an operator or journaling storage. The reliable scoring method may for example comprise an algorithm capable of enumerating the probability that a person is intoxicated. The enumeration may be displayable by some means as described herein enabling deployment in settings where some tolerance can be built in.

In some embodiments, the device comprises a reporting system to communicate results on the assessment of the intoxication status or level to an operator. The reporting system may for example, and without limitation, be a graphical or numerical reading expressed on a networked device, screen, matrix describer. The reporting system may also be an audible signal, such as for example a buzzer, sounds sample or any number of means for providing an audible alert. In an embodiment, the reporting system comprises a screen that graphically presents the one or more thermographic images, annotations to the one or more thermographic images, graphs, other suitable data, or any combination thereof to communicate the assessment of the intoxication status or level to the operator. In an embodiment, the reporting system comprises a matrix display, LCD, LED, buzzer, speaker, light, numerical value, picture, image, other visual or audio reporting means, or any combination thereof to communicate the assessment of the intoxication status or level to the operator.

In some embodiments, the device comprises one or more sensors or data inputs for recording date, time, position, orientation, temperature, humidity or other geo-temporal and physical conditions at the time of obtaining the one or more thermographic images.

In some embodiments, the device comprises one or more accessory components to ascertain identity of the operator(s) and/or the tested individual(s). In an embodiment, the one or more accessory components ascertain the identity of the operator(s) and/or the individual(s) by manual input, swipe card, barcode, biometric, RFID, NFC or other identifying means.

In some embodiments, the device comprises a switching apparatus to enable the device to operate in two or more different modes. In an embodiment, a first mode operates using a priori data on the individual, such as for example one or more non-intoxicated state thermographic images of the individual. In an embodiment, a second mode operates in the absence of a priori data on the individual.

In some embodiments, the device comprises a tuning mechanism to adjust a tolerance and/or a sensitivity of the device. In an embodiment, the tuning mechanism is on the device or is remotely located if the device has a network connection.

In some embodiments, the device comprises an external communication component that is capable of communicating with other systems for receiving information, storage, or further processing. In exemplary embodiments, the system is capable of transmitting data in binary or character formats, encrypted or unencrypted.

In some embodiments, the device disclosed herein is capable of storing and/or transmitting data in a form that prevents manipulation post sampling. In some embodiments, the form for transmission comprises segments of an original version of the one or more thermographic images, sample points, intermediate results, final results, environmental readings, or any combination thereof, to be integrated via a one-way algorithm into a unique identifier, record length descriptor and checksum, all of which requiring accuracy in order for the original data to be considered authentic. In some embodiments, the form for storage comprises storing data into local or remote encrypted storage systems.

In some embodiments, the device disclosed herein is capable of fingerprinting internal structures of the device in order to guarantee a level of integrity of the sampling and assessment algorithms and configuration. For example, in select embodiments, the device is capable of a two-way communication with one or more remote storage systems, the two-way communication performing encrypted communications in a manner that guarantees the authenticity of those data stored on the device, on the remote system(s), or any combination thereof.

The device as disclosed herein may take any suitable form. In an embodiment, the device is a portable device, a hand-held device, or a kiosk (e.g. a self-service intoxicant dispensing kiosk). In an embodiment, the kiosk can be semi-mobile, being on the back of a truck, trailer or the like. In an embodiment, the device is a stand-alone device for use as a non-invasive screening tool for determining whether the individual is permitted to operate a vehicle or machine.

In an embodiment, the device is capable of being integrated into a vehicle or machine, and when integrated can prevent the individual from operating the vehicle or machine based on the assessment of the intoxication status or level of the individual.

In some embodiments, the device is one that screens individuals prior to entry to a site that requires a low or zero threshold of intoxication (e.g. sobriety). For example and without limitation, the site may be a work site, a job site, a recreational site, or an entertainment venue.

In some embodiments, the device may be integrated into, or a component of, a hardware device that assesses intoxication status or levels of individuals using the hardware device.

In other aspects, the present disclosure relates to a system comprising the device as described herein and a remote data processing and/or storage component.

In other aspects, the present disclosure relates to a computer readable medium having recorded thereon executable instructions that when executed by a computer conduct an analysis of one or more thermographic images to provide an assessment of the intoxication status or level of the individual. In an embodiment, the computer readable medium executes analysis of the one or more thermographic images based on the methods disclosed herein (e.g. one or more of (i)-(ix) as described herein).

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Many obvious variations of the embodiments set out herein will suggest themselves to those skilled in the art in light of the present disclosure. Such obvious variations are within the scope of the appended claims.

1. A method for assessing an intoxication status of an individual, the method comprising the steps of:
receiving a thermographic image comprising a face or facial features of the individual;
performing pre-processing of the thermographic image to provide a pre-processed image;
identifying a face portion comprising the face or facial features in the pre-processed image; and
analyzing the face portion using an intoxication assessment method to assess the intoxication status.

2. The method of claim 1, wherein the thermographic image further comprises faces or facial features of other persons and the step of identifying the face portion comprises isolating the face or facial features of the individual.

3. The method of claim 1, wherein the pre-processing comprises:
reducing the thermographic image to a single channel; and/or
removing data from the thermographic image for temperatures outside of a temperature range.

4. (canceled)

5. The method of claim 3, wherein the temperature range is between 17 degrees Celsius and 47 degrees Celsius.

6. The method of claim 3, wherein the temperature range is between 32 degrees Celsius and 42 degrees Celsius.

7. The method of claim 1, wherein the pre-processing comprises reducing a resolution of the thermographic image.

8. (canceled)

9. The method of claim 1, wherein the step of identifying the face portion comprises using a Haar Cascade.

10. The method of claim 1, further comprising the step of detecting an obstruction to the face portion.

11. (canceled)

12. (canceled)

13. The method of claim 1, wherein the intoxication assessment method comprises:
identifying a plurality of points in the face portion to provide a facial feature vector;
comparing the facial feature vector with other facial feature vectors for other thermographic images to identify differences therebetween; and
assessing the intoxication status by analyzing the differences,
wherein the plurality of points comprises at least twenty.

14. (canceled)

15. The method of claim 1, wherein the intoxication assessment method comprises:
identifying at least two regions in the face portion; and
determining a face temperature difference between the two regions for assessing the intoxication status.

16. The method of claim 15, wherein the two regions comprise a nose area and a forehead area, the nose area comprising an image of a nose of the individual and the forehead area comprising an image of a forehead of the individual.

17. The method of claim 1, wherein the intoxication assessment method comprises:
identifying an eye region in the face portion, the eye region comprising an image of eyes of the individual;
identifying a sclera region and an iris region within the eye region, the sclera region comprising an image of a sclera of the individual and the iris region comprises an image of an iris of the individual; and
determining an eye temperature difference between the sclera region and the iris region for assessing the intoxication status.

18. (canceled)

19. The method of claim 1, wherein the intoxication assessment method comprises:
identifying a high correlation area in the face portion; and
using a trained neural network to analyze the high correlation area to assess the intoxication status.

20. The method of claim 19, wherein the high correlation area is a nose area, a mouth area or a combination thereof, wherein the nose area comprises an image of a nose of the individual and a mouth area comprises an image of a mouth of the individual.

21. The method of claim 1, wherein the intoxication assessment method comprises:
identifying blood vessel in the face portion; and
analyzing the blood vessels to determine changes or differences in blood vessel activity to assess the intoxication status.

22. The method of claim 21, wherein the step of analyzing blood vessel locations comprises applying a nonlinear anisotropic diffusion and a top-hat transformation of the face portion.

23. The method of claim 21, wherein the step of analyzing blood vessel locations comprises using image processing to perform image transformations, convolutions, edge detections, or related means to determine changes or differences in blood vessel activity.

24. The method of claim 1, wherein the intoxication assessment method comprises identifying and analyzing one or more isothermal regions in the face portion to assess the intoxication status.

25. The method of claim 24, wherein the step of identifying and analyzing one or more isothermal regions comprises determining a shape and a size of the isothermal regions.

26. The method of claim 24, wherein at least of one of the isothermal regions is a forehead region of the face portion comprising an image of a forehead of the individual, and wherein when the forehead region is thermally isolated from a remainder of the face portion, the intoxication status is intoxicated.

27. The method of any one of claims 1 to 26, wherein the intoxication assessment method comprises one or more of:
using Markov chains or Bayesian networks for modeling statistical behaviour of pixels in a forehead region of the face portion, wherein the forehead region comprises an image of a forehead of the individual;
identifying local difference patterns in the face portion to assess the intoxication status; and
feature fusion analysis to fuse dissimilar features of the face portion using neural networks to assess the intoxication status.

28.-31. (canceled)

32. A system for assessing an intoxication status of an individual, the system comprising a computer configured to:
perform pre-processing of one or more thermographic images comprising a face or facial features of the individual;
identifying a face portion of the thermographic images comprising the face or facial features; and
analyzing the face portion using at least one intoxication assessment method to assess the intoxication status.

33.-47. (canceled)

48. One or more non-transitory computer-readable storage devices comprising computer-executable instructions for providing an assessment of an intoxication status of an individual, wherein the instructions, when executed, cause a processing structure to perform actions comprising:
receiving a thermographic image comprising a face or facial features of the individual;
performing pre-processing of the thermographic image to provide a pre-processed image;
identifying a face portion comprising the face or facial features in the pre-processed image; and
analyzing the face portion using an intoxication assessment method to assess the intoxication status.

49.-56. (canceled)
